SEQUENCE LISTING (8 sequences)

SEQ ID NO: 1 - protein, 189 aa, human
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAGTERWGWGRGDATPIQGWAIGAVWRGGSAIGVLETVDQASTN

SEQ ID NO: 2 - protein, 173 aa, human
MAAPSGGWNGVGASLWAALLLGAVALRPAEAVSEPTTVAFDVRPGGVVHSFSHNVGPGDKYTCMFTYASQGGTNEQWQMSLGTSEDHQHFTCTIWRPQGKSYLYFTQFKAEVRGAEIEYAMAYSKAAFERESDVPLKTEEFEVTKTAVAHRPGAFKAELSKLVIVAKASRTEL

SEQ ID NO: 3 - protein, 83 aa, human
HQHFTCTIWRPQGKSYLYFTQFKAEVRGAEIEYAMAYSKAAFERESDVPLKTEEFEVTKTAVISAPQEKGDDQPHWTDGETEA

SEQ ID NO: 4 - DNA, 21 bp, artificial
  PCR primer
ggtgtccgagcccacgacggt

SEQ ID NO: 5 - DNA, 26 bp, artificial
  PCR primer
catggctcgagtcacagctcagtgcg

SEQ ID NO: 6 - DNA, 28 bp, artificial
  PCR primer
gaattccatatggtgtccgagcccacga

SEQ ID NO: 7 - DNA, 27 bp, artificial
  PCR primer
catggctcgagcagctcagtgcgcgat

SEQ ID NO: 8 - DNA, 26 bp, artificial
  PCR primer
catgcggatcccggtgtccgagccca

(19) United States
(12) Patent Application Publication
(10) Pub. No.: US 2008/0004232 A1
(43) Pub. Date: Jan. 3, 2008

(54) Characterization of c19orf10, a Novel Synovial Protein

(76) Inventors: John Wilkins, Manitoba (CA); Hani El-Gabalawy, Manitoba (CA); Oleg Krohkin, Manitoba (CA); Kumar Dasuri, Manitoba (CA); Quijiang Du, Manitoba (CA); Werner Ens, Manitoba (CA)

Correspondence Address: ADE & COMPANY INC., 2157 Henderson Highway, WINNIPEG, MB, R2G1P9, CA

(21) Appl. No.: 11/746,439

(22) Filed: May 9, 2007

Related U.S. Application Data
(60) Provisional application No. 60/746,828, filed on May 9, 2006.
(60) Provisional application No. 60/805,610, filed on Jun. 23, 2006.

(51) Int. Cl. A61K 31/7088; A61P 19/02; C12Q 1/68

(52) U.S. Cl. 514/44; 435/6

(57) ABSTRACT

Described herein is the first in-depth analysis of c19orf10. Immunofluorescence analysis of cultured cells suggests that the c19orf10 product is localized to the ER/golgi apparatus. The symbol tissue distribution is unique, with staining of both the synovial membrane and the perivascular regions. In some cases there also appears to be an inverse relationship between synovial hyperplasia and c19orf10 expression, raising the possibility that c19orf10 production may be lost during hyperplasia.

### PRIOR APPLICATION INFORMATION

This application claims the benefit of U.S. Provisional Patent Application 60/746,828, filed May 9, 2006 and U.S. Provisional Patent Application 60/805,610, filed Jun. 23, 2006.

### BACKGROUND OF THE INVENTION

The healthy synovial membrane consists of a thin layer of fibroblast-like synoviocytes (FLS) and macrophages. These cells produce glycosaminoglycans such as hyaluronic acid and lubricating glycoproteins for secretion into the synovial fluid [1]. Healthy homeostasis within the joint can be disturbed by development of inflammatory diseases such as rheumatoid arthritis (RA). In this situation, the synovium becomes enlarged and the cellular composition changes. The intimal layer exhibits an increased number of FLSs, there is an increase in the number of blood vessels, and the sub-intima becomes infiltrated with lymphocytes and plasma cells forming ectopic lymphoid follicles [2]. The phenotype of the synovial cells also changes. In vitro, RA-FLSs exhibit a transformed phenotype reminiscent of that seen in tumors (increased proliferative potential and resistance to apoptosis), whereas the vascular endothelium displays an increased ratio of apoptotic to proliferative cells, which is indicative of vascular remodeling [3]. These changes may contribute to the maintenance of synovial inflammation, aggravating the destruction of the cartilage and bone and encouraging the development of the pannus. Because FLSs can exhibit significant phenotypic changes under different pathological conditions, studies were initiated to examine the repertoire of proteins produced by these cells. Ultimately, these studies could provide information regarding differences in protein synthesis by FLS in health and disease.

Previously, proteomic studies were initiated to determine the protein composition and expression patterns of FLSs [4]. These studies resulted in 254 identifications corresponding to 192 distinct proteins. One of the proteins identified was a major FLS protein encoded by the novel gene, c19orf10 (chromosome 19 open reading frame 10). c19orf10 has been identified in two other reports in the literature. Tulin et al performed a genetic complementation screening approach looking for stromal cell-derived factors involved in cell proliferation [5] and identified c19orf10 as a murine bone marrow stroma-derived growth factor (SF20/IL-25). Subsequent work revealed that the proliferation data described in the initial report could not be replicated and was withdrawn [6]. In another report, Wang et al profiled the proteins secreted from pre-adipocytes (3T3-L1) using 2DE-MS [7]. A number of secreted proteins were identified including c19orf10 (SF20/IL25), which was up-regulated during differentiation of adipocytes, leading to the suggestion that it is involved in adipogenesis. While these studies have postulated that c19orf10 is involved in cell proliferation and differentiation, neither of these studies has revealed any functional information about the molecule. We undertook the characterization of c19orf10 in the synovium because (a) it is a quantitatively significant product of FLS [4], (b) little is know about processing and function of the c19orf10 molecule and (c) there was a lack of analytical reagents.

### SUMMARY OF THE INVENTION

According to a first aspect of the invention, there is provided a

### BRIEF DESCRIPTION OF THE DRAWINGS

FIG. 1. Proteomic identification of c19orf10. A) MS spectrum of in-gel digest of c19orf10 with six peptides mapping to the c19orf10 protein labeled. B-D) MS/MS spectra of three c19orf10 parent ions. Peaks contributing to the score are labeled. The mass and sequence of each parent ion is indicated on the appropriate spectrum.

FIG. 2. Genomic Organization, Alternative Splicing and Protein Sequence of c19orf10. A) The chromosomal localization of the region containing the c19orf10 gene is indicated on the ideogram. The area containing the c19orf10 gene is expanded and the base pair positions are indicated. The location of a microsatellite marker linked to juvenile rheumatoid arthritis, D19S216, is also indicated. B) The expanded region of chromosome 19 containing the c19orf10 gene. Three putative splicing variants are indicated (c19orf10.a, c19orf10.b, c19orf10.c). Thick blocks indicate translated exons, open blocks indicate untranslated exons, horizontal lines indicate introns and the arrows indicate the direction of transcription. C) Alignment of protein products of c19orf10 splicing variants. Variants a and b seem to be complete sequences starting with an N-terminal methionine. Variant c does not start with an N-terminal methionine and is probably incomplete at the N-terminus. Lines above the sequence map the exons to the protein sequence. Shaded sequences indicate peptides observed by MS. The putative N-terminal signal peptide is underlined with a solid black line. C63 & 92 are indicated by with rectangles.

FIG. 3. c19orf10 multiple sequence alignment. All sequences were obtained from Genbank. H. sapiens—NP_{—}061980; M. musculus—NP_{—}543027; R. norvegicus—XM_{—}347126; S. scrofa—SSC.4092; B. taurus—NP_{—}01001164; C. familiaris—ENSCAFT30192; G. gallus—NP_{—}001006342; D. rerio—NP_{—}001002480; T. nigroviridis—CAGO8012; L. erinacea—CV067465 (translated in reading frame +3); S. acanthias—CX789984 (translated in reading frame +2).

FIG. 4. c19orf10 Immunofluorescence. A,B,C,D) Immunofluorescence of early passage FLS. A,C) FLS were labeled with anti-c19orf10 monoclonal antibody, 1B6, and visualized using red fluorescent Cy3 goat-anti mouse IgG (H&L) antibody. The cells were counterstained with green fluorescent Oregon Green phalloidin to visualize the F-actin; B,D) Negative control with no primary antibody, stained as above.

FIG. 5. Expression of c19orf10 in RA and OA synovium. A,D,F,H) Expression of c19orf10 in RA synovium. B,C,E,G) Expression of c19orf10 in OA synovium. A,B) Intense staining of the synovial lining layer and perivascular regions of both RA (OCT section) and OA (paraffin section) tissues. C) An area demonstrating a thin lining layer and perivascular region populated with c19orf10 positive cells. Note that the sublining stroma in this area is virtually devoid of c19orf10 staining. This pattern of staining is typical of that seen in both RA and OA sections. D) In most lymphocytic aggregates, there was minimal staining of the lymphocytes, although some mononuclear cells in the aggregates stained positively (arrow). E) Intense staining of individual cells in the lining layer of a typical osteoarthritis synovium. F) An area of an OA synovium demonstrates a lining layer completely devoid of c19orf10 staining. G) Intense staining of a hyperplastic RA synovial lining cell layer. This staining was typical of most areas of RA synovium where the lining was hyperplastic. H) An area of an RA synovial lining layer that is not positive for c19orf10 staining.

FIG. 6. Demonstration of c19orf10 in synovial fluid. Synovial fluid c19orf10 levels were determined for five patients with the indicated arthropathies by competitive ELISA. Each fluid was measured at two different dilutions. The concentrations for each are indicated in the table. A representative standard curve is presented in the graph.

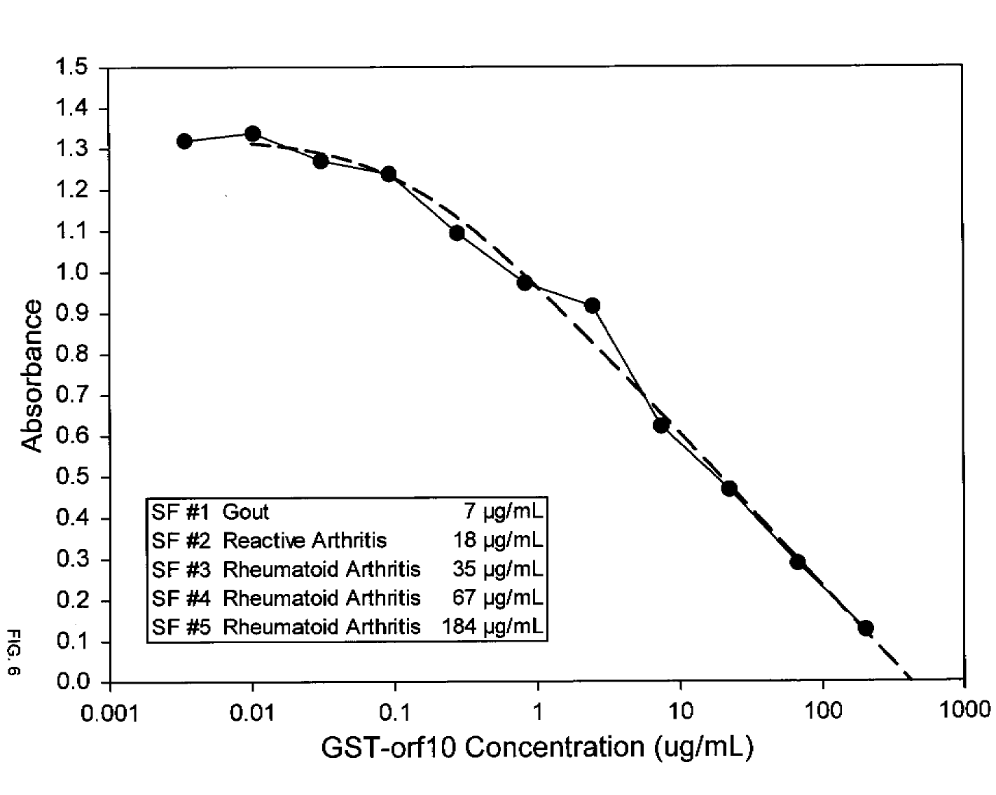

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Described herein is the first in-depth analysis of c19orf10 (SEQ ID NOs 1-3). In silico analysis of the sequence indicates the presence of a leader peptide; however, it does not suggest a function for this protein. Proteomic analysis of the molecule suggests that the putative leader peptide is cleaved and the mature protein is secreted out of the cell. A multiple sequence alignment of c19orf10 homologues reveals a high degree of similarity amongst vertebrates. Immunofluorescence analysis of cultured cells suggests that the c19orf10 product is localized to the ER/golgi apparatus. The synovial tissue distribution is unique, with staining of both the synovial membrane and the perivascular regions. In some cases there also appears to be an inverse relationship between synovial hyperplasia and c19orf10 expression, raising the possibility that c19orf10 production may be lost during hyperplasia.

Joint inflammation and destruction has been linked to the deregulation of the highly synthetic fibroblast-like synoviocytes (FLS) and much of our current understanding of the mechanisms that underlie synovitis has been collected from studies of FLSs. During a proteomic analysis of FLS cells, we identified a novel protein, c19orf10 (chromosome 19 open reading frame 10) that was produced in significant amounts by these cells. The present study provides a partial characterization of c19orf10 in FLSs, synovial fluid, and the synovium. Murine monoclonal and chicken polyclonal antibodies were produced against recombinant human c19orf10 protein and used to examine the distribution of c19orf10 in cultured FLSs and in synovial tissue sections from patients with rheumatoid arthritis or osteoarthritis. The intracellular staining patter of c19orf10 is consistent with localization in the endoplasmic reticulum/golgi distribution. Sections of rheumatoid arthritis and osteoarthritis synovia expressed similar patterns of c19orf10 distribution with perivascular and synovial lining staining. Double-staining in situ analysis suggests that fibroblast-like synovial cells produced c19orf10, whereas macrophages, B cells, or T cells produced little or none of this protein. There is evidence of secretion into the vascular space and the extracellular matrix surrounding the synovial lining. A competitive enzyme-linked immunosorbent assay confirmed the presence of microgram levels of c19orf10 in the synovial fluids of patients with one of various arthropathies. Collectively, these results suggest that c19orf10 is an FLS-derived protein that is secreted into the synovial fluid.

The intensity of the c19orf10 spot on the 2DE gel suggests that the protein is relatively abundant in the FLS. MS/MS analysis of the spot corresponding to c19orf10 indicated 6 peptides matched to the sequence with coverage of 47% and a GPM log(e) value of −16.8. A multiple sequence alignment indicates a high degree of identity among vertebrates. IF analysis of FLS indicates that c19orf10 is localized in a pattern consistent with ER/Golgi distribution and a competitive ELISA assay has confirmed the presence of high levels of c19orf10 in the synovial fluid. IHC analysis of frozen and paraffin-embedded sections of both RA and OA synovium reveals a unique pattern of perivascular and synovial lining staining. There is evidence of secretion into the vascular space and the extracellular matrix surrounding the synovial lining. Furthermore, IHC staining is absent in some regions of hyperplasia. These observations suggest that C19ORF10 levels/production may be altered in cells that are growing or dedifferentiated. This correlation may be useful in diagnosis and suggests a role for C19ORF10 in the prevention of these changes.

Our results suggest that c19orf10 is expressed relatively abundantly in the FLS. It is secreted into the synovial fluid, consistent with the presence of a signal peptide, the identification of a non-tryptic signal peptide cleavage site, and ER/Golgi distribution. Distribution of c19orf10 immunoreactivity seems to vary with the degree of cellularity, suggesting that subsets of FLS exhibit differences in the production or functional capability of c19orf10. Assay of C19ORF10 in synovial, plasma or urine may provide a method of detecting synovial changes and/or monitoring responses to therapy (i.e. knowing the normal range and following returns to baseline during treatment). Levels of expression may also be useful in defining categories or types of arthropathies for diagnostic and treatment purposes.

The intracellular distribution of c19orf10 staining suggests that the protein may be processed for secretion in the ER/Golgi compartments. This is consistent with the prediction that c19orf10 has a leader sequence. Furthermore, the MS data indicated that there was a non-tryptic cleavage at the predicted interface between the leader and the mature protein. The synovial lining distribution of the c19orf10 staining is also compatible with this protein being secreted, as type b synovial fibroblasts are responsible for the production and secretion of other components of synovial fluid. The results of the competitive c19orf10 ELISA indicated that the fluid concentrations ranged from 0.5 to 12 mM. This represents a significant level even at the lower concentration but these values are consistent with those reported for synovial fluid levels of MMP3 and TIMP1 [14].

The c19orf10 protein may be broadly expressed. High levels of gene expression were observed in testis, spleen, and heart with moderate levels in the lung and liver. In contrast brain, kidney and skeletal muscle did not show any expression. These results may suggest a general role in tissue related secretory processes and as such may expand the applicability range of C19ORF10 and the value in monitoring in other conditons. Significant expression levels were found in carcinomas of the lung, breast and colon, while respective normal tissues were negative. Resting CD8+ cells, CD19+ and mononuclear cells were shown to have significant c19orf10 expression. Conversely, resting CD4+ and CD14+ cells were negative for c19orf10 as were activated CD19+ cells, CD4+ cells and mononuclear cells. The proteomic studies of Wang et al identified c19orf10 as a secreted product of 3T3 fibroblasts as they differentiate into adipocytes [7]. Collectively these observations suggest that c19orf10 may not be unique to the synovial compartment; however, the results of the present study clearly indicate that it is a significant synovial fluid component at least in disease states.

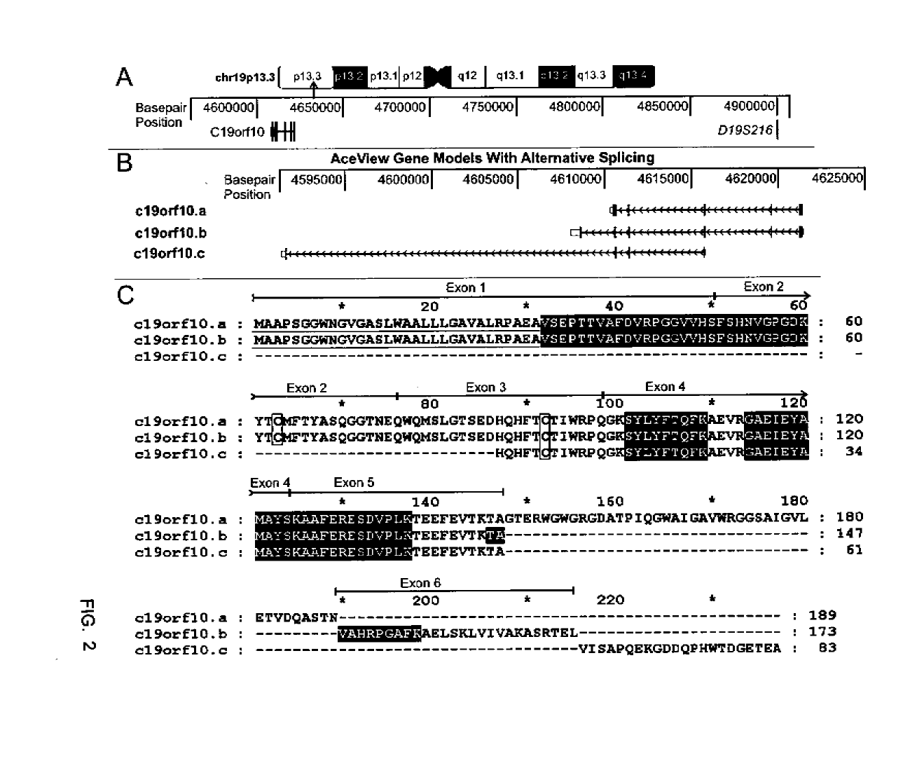

Regarding FIG. 2, as can be seen, exons 3, 4 and 5 are common to all three variants and accordingly in one embodiment of the invention, there is provided a purified or isolated peptide comprising amino acids 76-147 of SEQ ID No. 2. Similarly, amino acids 1-147 are common to variants a and b. As discussed below, c19orf10 has a leader sequence and accordingly, in some embodiments, the peptide comprises amino acids 32 to 173 of SEQ ID No 2.

In one embodiment of the invention, there is provided a method of determining if a tissue is undergoing altered growth comprising measuring c19orf10 expression levels in the tissue wherein c19orf10 expression levels below a threshold level indicates that the tissue is undergoing altered growth, for example, abnormal growth. The threshold level may be an average determined by measurement of c19orf10 levels in a number of subjects of similar age and condition or may be determined by measuring c19orf10 levels in other tissues of the same subject.

In another aspect of the invention, there is provided a method of monitoring changes in a tissue comprising determining c19orf10 expression level in the tissue at a first time point; and determining c19orf10 expression level in the tissue at a second time point. As will be appreciated by one of skill in the art, changes in c19orf10 expression levels over time can thus be used to monitor changes as a result of treatment or caused by disease progression or for classifying a disease.

In another aspect of the invention, there is provided a method of treating a joint comprising administering to the joint an effective amount of a c19orf10 peptide.

In another aspect of the invention, there is provided a method of treating a joint comprising administering to the joint an effective amount of a peptide comprising amino acids 32 to 173 of SEQ ID No. 2. In other embodiments, an effective amount of a peptide consisting essentially of or consisting of amino acids 32 to 173 of SEQ ID No. 2 may be administered.

As will be appreciated by one of skill in the art, the tissue may be synovial tissue.

It is of note that methods for determining c19orf10 expression levels, either transcript levels and/or peptide levels, are well known in the art and many examples by which this may be done are described herein. It is also of note that total c19orf10 levels may be determined by using probes directed to regions common to all three known variants as discussed above while expression of specific variants may be determined by using probes specific for unique regions, for example, the C-terminal regions.

It is interesting to note that c19orf10 is localized to chromosome 19p13.3. This region also contains the microsatellite marker, D19S216, a marker that is reported to be linked to a juvenile rheumatoid arthritis susceptibility (JRA) locus [15]. Specifically, the pauciarticular course of JRA is linked to this 19p13 marker with a LOD score of 2.98. In addition, this marker was also linked to the subset of patients showing pauciarticular onset of disease. c19orf10 is located approximately 300 kb upstream of D19S216, making it a candidate locus for the JRA phenotype.

We have chosen not to assign a name to the c19orf10 product at this time because there is no clear understanding of the function of this protein. The previous name IL-25 was based on an apparent growth factor activity [6]. The claim of this activity was subsequently retracted and this has led to significant confusion in the literature and the databases regarding the designation IL-25. c19orf10 does not show any similarity to either IL-25 or IL-27. The IL-25 annotation has been changed in the NCBI databases and HGNC has indicated that the designations IL25 and IL27 will not be used.

The protein encoded for chromosome 19 open reading frame 10 is produced and secreted by FLS. This protein is a major component of synovial fluids in patients with various arthropathies. The tissue patterns of synthesis may change with synovial tissue cellularity suggesting either changes in production rates or capabilities may occur in subsets of synovial fibroblasts in RA and OA. Assay of C190RF10 in synovial, plasma or urine may provide a method of detecting synovial changes and/or monitoring responses to therapy (i.e. knowing the normal range and following returns to baseline during treatment). Levels of expression may also be useful in defining categories or types of arthropathies for diagnostic and treatment purposes. For example, it may be particularly relevant in OA which at this time has unknown pathogenic mechanisms and lacks good assays for prediction and monitoring.

Results

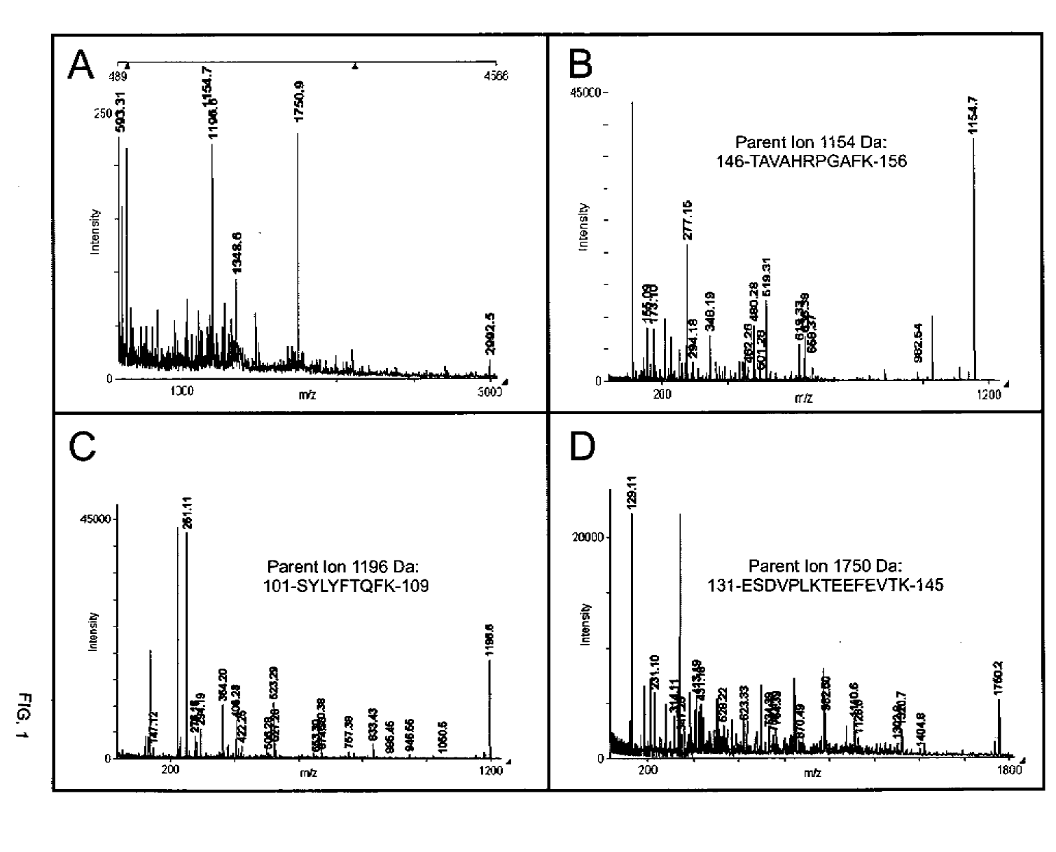

Proteomic analysis of the peptides obtained from a spot on a 2DE gel identified c19orf10 with a molecular weight of 16.5 kDa [4]. Six peptides mapped to the c19orf10 sequence with coverage of 47% (FIG. 1A). The Profound expectation score was 2.5×10⁻⁶[11]. Subsequent MS/MS analysis of the parent ions from three of the six peptides confirmed the identification with a GPM log (e) score of −16.8 (FIGS. 1B-D) [12]. Furthermore, manual MS/MS analysis of the 2992.5 Da parent ion indicated that it resulted from a non-tryptic peptide corresponding to amino acids 32-60.

The c19orf10 gene is located on chromosome 19p13.3 spanning approximately 30 kb (FIG. 2). A survey of the available cDNA clones suggests that three splice variants for c19orf10 (FIG. 2B). The most common variant, c19orf10. b (SEQ ID No. 2), has six exons and is supported by sequence data from 468 clones. The two other variants (a (SEQ ID No.1), c (SEQ ID No. 3)) have been identified with sequence data support from one clone each. It is likely that variant c is incomplete at the 5′ end. Protein products of the three splice variants are illustrated in FIG. 2C. All of the c19orf10 peptides identified by our MS analysis can be found in the c19orf10.b sequence (highlighted peptides, FIG. 2C), suggesting that c19orf10.b is the predominant molecular variant present in the synovial fibroblasts. It is possible that the other splice variants may also be present in these samples.

The c19orf10 gene product was predicted to be a 173 amino acid protein with a theoretical molecular weight of 18.8 kDa and a theoretical pl of 6.2. Two cysteine residues, at positions 63 and 92, (FIG. 2C—outlined with a rectangle) were predicted to be disulfide bonded. A 31 amino acid signal peptide was predicted using the SignalP and PSORTII algorithms (FIG. 2C—underlined), suggesting that this protein is secreted from the cell. This result is consistent with our MS analysis, lending support to the prediction that this was a secreted protein. In silico pattern analysis of the human c19orf10 sequence indicated that there may be two O-glycosylation sites at threonine positions 36 and 37, however the presence of the 2992.5 Da peak in the mass spectrum corresponding to non-glycosylated amino acid residues 32-60 suggested that at least a portion of the protein is not glycosylated. In addition, there are eight potential phosphorylation sites predicted by the NetPhos 2.0 server: S33, S84, S132, S169, T36, Y61, Y67 and Y119.

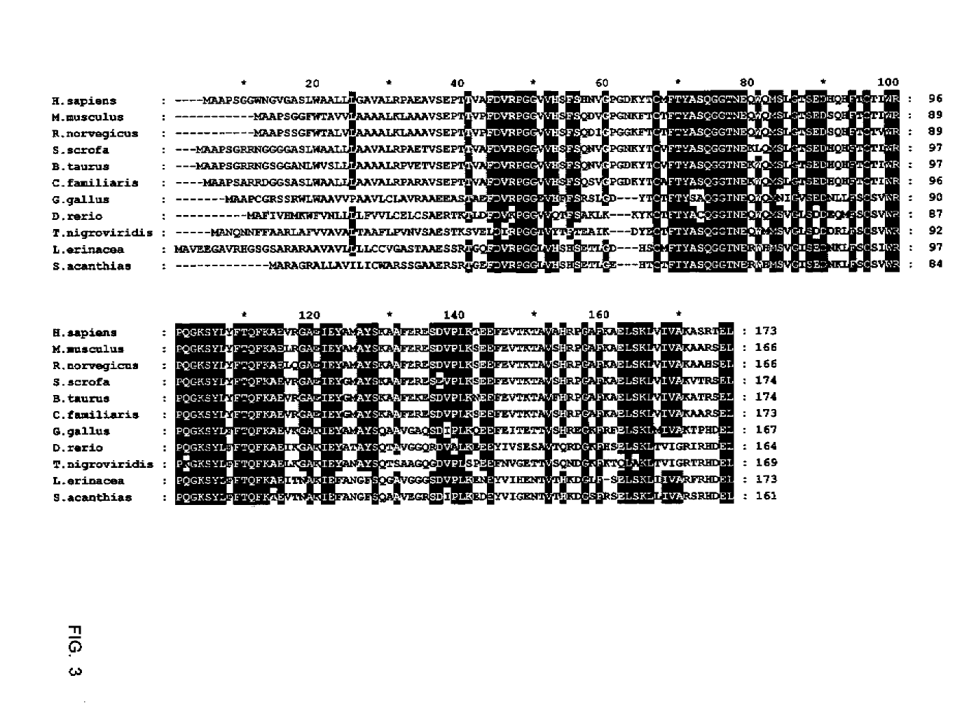

Nucleotide, protein and genomic databases from NCBI were searched for c19orf10 homologues. Sequence homologues were identified for 18 species of vertebrates including mammals, amphibians, birds and fishes. The c19orf10 aliases identified in the databases include R33729 (EMBL|CAB96948.1) and lymphocyte antigen 6 complex, locus E ligand (ly6e, GBINP_{—}543027.1). Several of the sequences are not full length and were omitted from further analyses. A multiple alignment of the remaining sequences was performed using the T-Coffee server (FIG. 3). The sequences were very similar in size, ranging from 161 to 174 amino acids. The N-terminal 40 residues in the area of the putative signal peptide were poorly conserved although the majority of the residues were non-polar. The remainder of the protein showed a high degree of conservation between the human sequence and all other sequences (FIG. 3). The two cysteine residues at positions 63 and 92 of the human sequence were completely conserved supporting the prediction that they participate in a disulfide bond. 28% of the residues are identical across the species listed and an additional 19% of the residues are highly conserved, suggesting that this protein has an important role in vertebrates.

A panel of murine monoclonal antibodies was produced against recombinant c19orf10 for immunohistochemistry and immunoassays in an effort to define the distribution of c19orf10 in synovium. Hybridomas were selected based on reactivity with c19orf10. All of these antibodies were IgM class. This may be attributable to the fact that there is 91% identity between the predicted mature human and murine proteins. Thus the immunogenicity was very low and an isotype switch was not achieved. Despite this fact, the antibodies were very effective in immunofluorescence as well as immunohistochemistry on both frozen and paraffin sections.

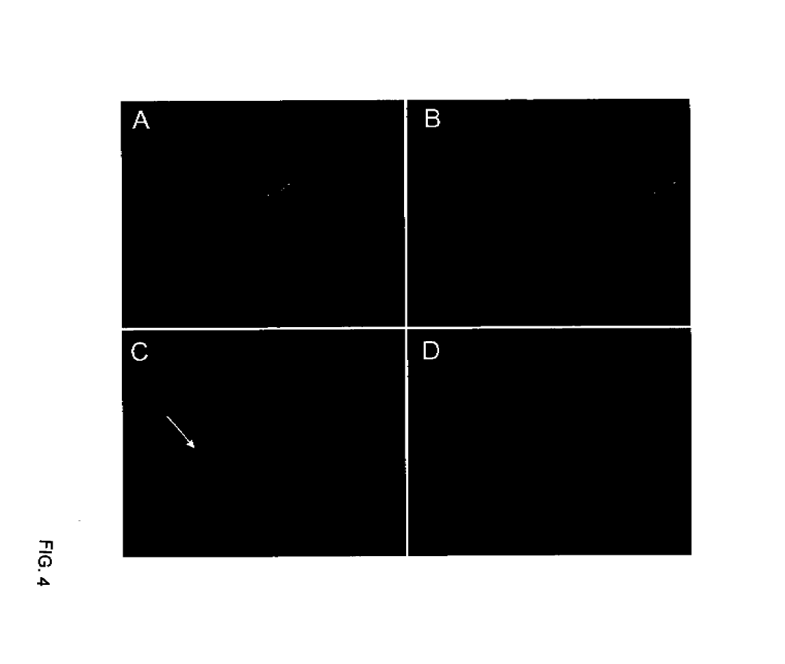

The staining of permeabilised low passage FLSs with the monoclonal antibody, 1B6, revealed a perinuclear punctate distribution, which was consistent with an ER/golgi distribution (FIGS. 4A,C). This staining pattern was detected with other antibodies to c19orf10 but not with control antibodies (FIG. 4B). In several cases there appeared to be a faint staining on the substrate surrounding the cells (FIG. 4C, arrow) suggesting that the protein was secreted.

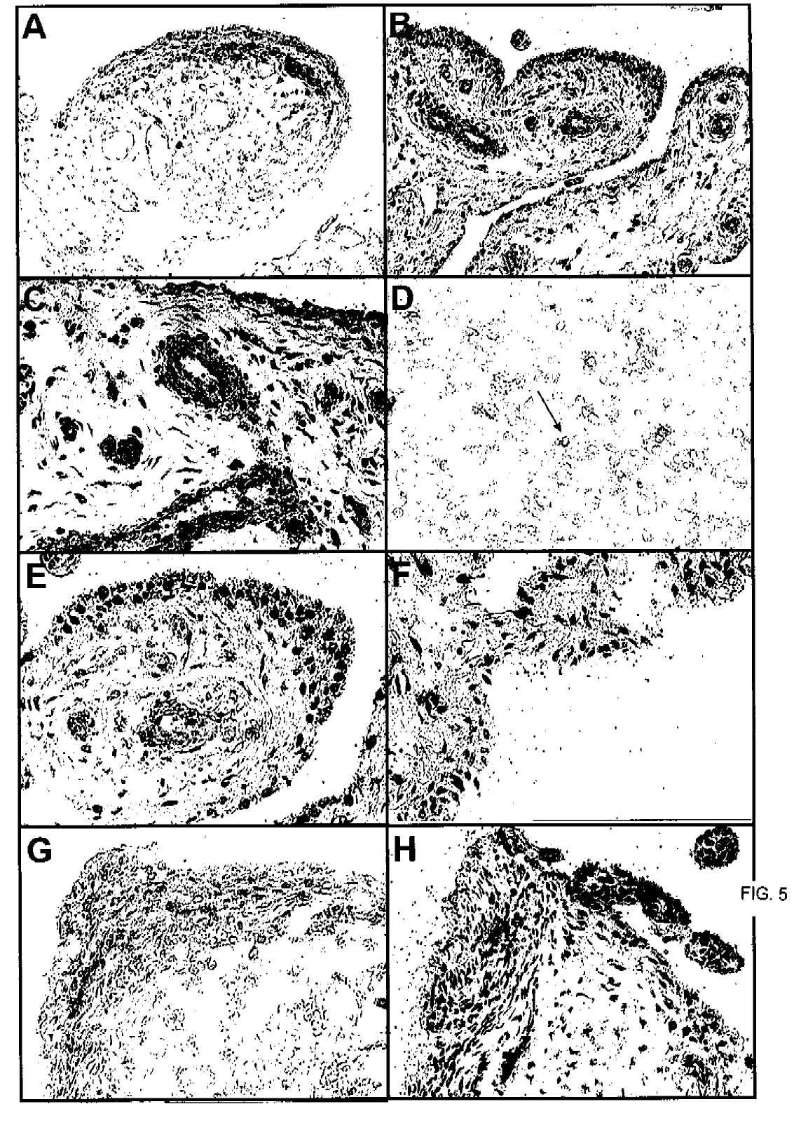

The c19orf10 distribution in synovial tissues from RA and OA patients was examined by immunohistochemistry. It was noteworthy that the tissues from both RA and OA patients displayed similar patterns of c19orf10 distribution (FIG. 5). At the gross level (FIGS. 5A, B), the majority of staining was in the synovial lining with some cells interspersed in the deeper tissues. Staining in the perivascular regions of the small vessels was also noted (FIG. 5C). In these cases, the staining appeared to be largely extracellular, suggesting that the secreted protein was bound at these sites. In regions of high mononuclear cellularity (FIG. 5D), there was intermittent staining of a subset of cells. In addition, areas of synovial hyperplasia showed variable staining patterns. In some though not all cases, these areas of hyperplasia were positive for c19orf10 staining (FIGS. 5E,G). In other cases, there was a marked absence of c19orf10 staining (FIGS. 5F, H). These observations raised the possibility that there may be either functional or compositional changes in the synovial cellular content which result in altered synthesis of this protein.

The cellular origins of the synovial tissues were examined using double-staining for c19orf10 and either CD68 as a marker for macrophage-like cells or CD59 as a marker for fibroblasts. There is clear colocalization of CD59 and c19orf10 staining throughout the synovial lining and in the underlying tissues. This contrasts with the situation with CD68, in which there was no obvious association between the bulk of c19orf10 distribution and the presence of CD68, suggesting that these cells are not the major producers of c19orf10 in the tissues examined. Similarly, staining for B cells (CD20) and T cells (CD25) also failed to show any codistribution with c19orf10.

The immunohistochemical results suggested that the synovial lining cells were producing significant levels of c19orf10. It was also noted in some sections that there appeared to be an accumulation of immunoreactive material on the lumenal surface of the synovium suggesting that c19orf10 was being secreted into the joint space. In order to test this possibility we developed an ELISA assay to monitor the synovial fluid levels of the protein. Initial assays with the murine IgM hybridomas were unsatisfactory, so chicken polyclonal antibodies were produced. Because chicken has a more distant phylogenetic relationship to humans, there was a greater possibility of obtaining high affinity antibodies with greater coverage. A hen was immunized with c19orf10-his fusion protein and the reactivity and specificity of the purified IgY was confirmed using GST-c19orf10 and a panel of unrelated His and GST fusion proteins. The antibody was then used to establish a competitive ELISA for the detection of c19orf10 in synovial fluids. The synovial fluids from five patients with different arthropathies were examined for the presence of c19orf10. The levels ranged from 7-184 μg/mL. This corresponds to millimolar concentrations of the protein. Specificity controls confirmed that only c19orf10 was detected in this assay, lending further support to the contention that this protein represented a major component of synovial fluid.

Materials and Methods

Cells, Tissues and Synovial Fluid

Synovial tissue was obtained from RA and osteoarthritis (OA) patients at the time of joint surgery. All RA patients met American College of Rheumatology criteria [8] and were receiving knee or hip arthroplasty. FLSs were isolated and cultured as previously described [9]. Synovial fluid was obtained from five patients diagnosed with rheumatoid arthritis, reactive arthritis or gout.

Sample Preparation, 2DE Analysis, In-Gel Digestion and Mass Spectrometry

Cell lysates were prepared and isolated as previously described [4]. The preparative 2DE and mass spectrometry was performed and described by Dasuri et al [4]. Digests were analyzed using MALDI QqTOF mass spectrometer [10] and proteins were identified by single MS (peptide mass fingerprinting) using ProFound [11] and by MS/MS using the Tandem search engine [12]. The National Center for Biotechnology non-redundant human database was used in both cases.

cDNA Cloning & Expression Constructs

The c19orf10 IMAGE clone (4562455) corresponding to GenBank accession number NM_{—}019107 was obtained from Open Biosystems (Huntsville, Ala.). A series of oligonucleotide primers were designed to amplify the coding region of the c19orf10 sequence (amino acids 32V-173L, excluding a putative N-terminal signal sequence). Primers Orf10F (GGTGTCCGAGCCCACGACGGT, SEQ ID No. 4) and Orf10R1 (catggctcgaGTCACAGCTCAGTGCG, SEQ ID No. 5) were used to amplify a 431 bp sequence of c19orf10 encompassing nucleotides 162-592, excluding the region coding for the putative N-terminal signal peptide but including the 3′ stop codon. This sequence was inserted into the PshA1 and XhoI sites of the pET41b vector (Novagen) resulting in a GST-His-c19orf10 fusion gene. Primers Orf10NdeI (gaattccatatGGTGTCCGAGCCCACGA, SEQ ID No. 6) and Orf10R2 (catggctcgagcAGCTCAGTGCGCGAT, SEQ ID No. 7) were used to amplify a 426 bp sequence of c19orf10 encompassing nucleotides 162-587, excluding both the region coding for the putative N-terminal signal peptide and the 3′ stop codon. This sequence was inserted into the NdeI and XhoI sites of the pET41b vector (Novagen) resulting in a c19orf10-His fusion gene. Primers Orf10BamHI (catgcggatccCGGTGTCCGAGCCCA, SEQ ID No. 8) and Orf10R1 (catggctcgaGTCACAGCTCAGTGCG, SEQ ID No. 9) were used to amplify a 431 bp sequence of c19orf10 encompassing amino acids 32V-173L, excluding the putative N-terminal signal peptide and including the C-terminal stop codon. This sequence was inserted into the BamHI and XhoI sites of the pGEX-5X-2 vector (Amersham) resulting in a GST-c19orf10 fusion gene. Restriction enzyme analysis and DNA sequencing were used to confirm the fidelities of the plasmids. The expression constructs were then transformed into Escherichia coli strain BL21 or the Rosetta2 (DE3) strain enhanced with 7 human tRNA genes (Novagen).

Recombinant protein expression was induced with 1 mM isopropyl-β-D-thiogalactopyranoside (Novagen) for 3 hrs at 37° C. The cells were subsequently collected and frozen overnight. The frozen cell pellet was resuspended in BugBuster reagent (Novagen) in 1×PBS and the cells were lysed by treatment with lysozyme (Sigma) and benzonase (Novagen) for 30 min at room temperature and then centrifuged at 15000 g for 20 min. The supernatant was collected and applied to a Sepharose nickel affinity column (Novagen) or a Sepharose GST affinity column (Novagen) as per manufacturer's instructions. Recombinant c19orf10-his tag was eluted from the Sepharose nickel affinity column with 300 mM imidazole. Recombinant GST-tagged c19orf10 was eluted from the glutathione column with 10 mM reduced glutathione. Unlabelled c19orf10 (aa 32-173, SEQ ID No. 10) was produced by digestion of GST-c19orf10 with Factor Xa for 4 hrs at room temperature and then purified through another GST affinity column. The released recombinant protein was collected in the effluent. Protein quality and purity was assessed by 12% SDS-PAGE and visualized with Imperial purple protein stain (Pierce).

Antibody Production

Monoclonal antibodies were generated as previously described [13]. Female Balb/c mice were immunized with recombinant human c19orf10 fusion protein containing both GST and His tags (GST-His-c19orf10) or c19orf10-his. 10 or 25 μg of c19orf10 fusion protein was mixed with Titer-Max Gold adjuvant (Cedarlane) and used to immunize subcutaneously. The mice were re-immunized one month later. Three months later, one mouse was re-immunized intraperitoneally without adjuvant four days prior to the fusion. After the fusion, hybridomas that were ELISA positive to c19orf10 were picked for further analysis and cloning. Cloned hybridoma cell lines were grown to death in RPMI-1640 containing 10% fetal bovine serum and supernatants were collected and used as a source of antibodies. The antibodies were characterized using the Isotyping Monoclonal Antibodies Kit. One of the anti-rhc19orf10 clones, 1B6, was grown in serum-free media and then purified using a KaptivM column (BioCan Scientific).

ELISA experiments were performed as previously described. Nunc Maxisorp multi-well plates were coated with 10 μg/mL c19orf10-His fusion protein overnight at 4° C. Plates were washed and then blocked with 1% BSA in PBS. The plates were incubated either with primary antibodies obtained from supernatant containing 10% serum or purified antibody. The plates were incubated with goat anti-mouse IgG (whole molecule) alkaline phosphatase conjugate (Sigma). ELISA response was detected with p-nitrophenyl phosphate substrate tablets (Sigma) and read at 405 nm on an ELISA plate reader.

A chicken polyclonal antibody was obtained by immunization with recombinant his-tagged c19orf10 (Gallus Immunotech, Inc). One hen was immunized twice with 100 μg of orf10-his and then twice with 50 μg of orf10-his. Immune eggs were collected and egg yolk IgY was purified by Gallus Immunotech, Inc. Competitive ELISA experiments were performed as follows. Nunc Maxisorp multi-well plates were coated with 0.8 μg/mL GST-c19orf10 fusion protein overnight at 4° C. Plates were washed and then blocked with 1% BSA in PBS. The plates were incubated either with anti-c19orf10 IgY or anti-c19orf10 IgY pre-incubated with GST-c19orf10 or synovial fluid. The plates were washed and then incubated with donkey anti-chicken IgY HRP conjugate (1:15000; Gallus Immunotech). ELISA response was detected with tetramethylbenzidine ELISA substrate solution (Sigma), stopped with 2 N sulfuric acid and read at 450 nm on an ELISA plate reader.

Immunofluorescence

FLSs were stained with monoclonal anti-rhc19orf10 antibodies. Twenty-one spot microscope slides (Erie Scientific) coated with 5 or 10 μg/mL fibronectin (Sigma) were seeded with 5×10⁴ cells/spot and incubated overnight in 37° C. with 10% CO₂. After incubation, the cells were washed in PBS and then fixed in 4% paraformaldehyde (PolySciences) in PBS for 15 min. The cells were then washed and in some cases permeabilised using 0.2% Triton X-100 (Sigma) in PBS for 5 min and washed again. The slides were treated with supernatants containing anti-rhc19orf10 antibodies or purified antibody for 1 hr at room temperature. Excess primary antibody was removed with 3 washes of PBS and the cells were then treated with Oregon Green 488 conjugated phalloidin (Invitrogen—Molecular Probes) in combination with cyanine-3 conjugated goat anti-mouse immunoglobulin (Jackson Immunoresearch). Fluorescence signal was visualized using an epifluorescence microscope (Olympus BX60) equipped with a xenon arc lamp, light pipe (Sutter Instruments, Lambda LS), and a Sensicam digital camera. Images were processed with Image Pro software (Media Cybernetics).

Immmunohistochemistry

Fresh frozen synovial biopsies were sectioned at 5 μm using a cryostat. Two sequential sections were picked up side-by-side onto a charged microscope slide (Fisher, ProbOn). Tissue staining was carried out using DAKO's ABC system. Slides with tissue sections were fixed in chilled acetone, air dried and re-hydrated in PBS. Endogenous tissue peroxidase was blocked by incubating the sections with hydrogen peroxide solution (DAKO). Sections were also blocked by incubation with normal serum from the animal species used for secondary antibody generation. Primary antibodies were then added to each tissue section and incubated overnight at 4° C. in a humidified slide chamber. The appropriate biotinylated secondary antibody (DAKO), streptavidin-horseradish peroxidase (DAKO), and diaminobenzidine substrate (DAKO) were utilized to detect the binding of the primary antibodies. Murine immunoglobulin of irrelevant specificity was added to a tissue section adjacent to the primary antibody and used as a negative control.

Paraffin blocks were prepared by immersing tissue samples in neutral buffered 10% formalin for 8 hours for fixation. They were dehydrated in ascending graded ethanol and infiltrated and embedded in low-melting paraffin at 56° C. in a heated oven. The tissue-paraffin mold was solidified on a cold plate to form a block. Four μm sections were cut by a microtome. The slides were then rehydrated in descending graded ethanol and processed through the same immunostaining steps as for frozen sections.

While the preferred embodiments of the invention have been described above, it will be recognized and understood that various modifications may be made therein, and the appended claims are intended to cover all such modifications which may fall within the spirit and scope of the invention.

### REFERENCES

1. Firestein G S: Invasive fibroblast-like synoviocytes in rheumatoid arthritis. Passive responders or transformed aggressors? Arthritis Rheum. 1996, 39:1781-1790.
2. Middleton J, Americh L, Gayon R, Julien D, Aguilar L, Amalric F and Girard J P: Endothelial cell phenotypes in the rheumatoid synovium: activated, angiogenic, apoptotic and leaky. Arthritis Res. Ther. 2004, 6:60-72.
3. Funk J L, Wei H, Downey K J, Yocum D, Benjamin J B and Carley W: Expression of PTHrP and its cognate receptor in the rheumatoid synovial microcirculation. Biochem. Biophys. Res. Commun. 2002, 297:890-897.
4. Dasuri K, Antonovici M, Chen K, Wong K, Standing K, Ens W, El-Gabalawy H and Wilkins J A: The synovial proteome: analysis of fibroblast-like synoviocytes. Arthritis Res. Ther. 2004, 6:R161-8.
5. Tulin E E, Onoda N, Nakata Y, Maeda M, Hasegawa M, Nomura H and Kitamura T: SF20/IL-25, a novel bone marrow stroma-derived growth factor that binds to mouse thymic shared antigen-1 and supports lymphoid cell proliferation. J.Immunol. 2001,167:6338-6347.
6. Tulin E E, Onoda N, Nakata Y, Maeda M, Hasegawa M, Nomura H and Kitamura T: SF20/IL-25, a novel bone marrow stroma-derived growth factor that binds to mouse thymic shared antigen-1 and supports lymphoid cell proliferation. J. Immunol. 2003, 170:1593.
7. Wang P, Mariman E, Keijer J, Bouwman F, Noben J P, Robben J and Renes J: Profiling of the secreted proteins during 3T3-L1 adipocyte differentiation leads to the identification of novel adipokines. Cell Mol.Life Sci. 2004, 61:2405-2417.
8. Arnett F C, Edworthy S M, Bloch D A, McShane D J, Fries J F, Cooper N S, Healey L A, Kaplan S R, Liang M H and Luthra H S: The American Rheumatism Association 1987 revised criteria for the classification of rheumatoid arthritis. Arthritis Rheum. 1988, 31:315-324.
9. Hitchon C, Wong K, Ma G, Reed J, Lyttle D and El-Gabalawy H: Hypoxia-induced production of stromal cell-derived factor 1 (CXCL12) and vascular endothelial growth factor by synovial fibroblasts. Arthritis Rheum. 2002, 46:2587-2597.
10. Loboda A V, Krutchinsky A N, Bromirski M, Ens W and Standing K G: A tandem quadrupole/time-of-flight mass spectrometer with a matrix-assisted laser desorption/ionization source: design and performance. Rapid Commun.Mass Spectrom. 2000,14:1047-1057.
11. Zhang W and Chait B T: ProFound: an expert system for protein identification using mass spectrometric peptide mapping information. Anal. Chem. 2000, 72:2482-2489.
12. Craig R and Beavis R C: TANDEM: matching proteins with tandem mass spectra. Bioinformatics 2004, 20:1466-1467.
13. Weiler T, Sauder P, Cheng K, Ens W, Standing K and Wilkins J A: A proteomics-based approach for monoclonal antibody characterization. Anal.Biochem. 2003, 321:217-225.
14. Higuchi H, Shirakura K, Kimura M, Terauchi M, Shinozaki T, Watanabe H and Takagishi K: Changes in biochemical parameters after anterior cruciate ligament injury. Int.Orthop. 2006, 30:43-47.
15. Thompson S D, Moroldo M B, Guyer L, Ryan M, Tombragel E M, Shear E S, Prahalad S, Sudman M, Keddache M A and Brown W M, et al: A genome-wide scan for juvenile rheumatoid arthritis in affected sibpair families provides evidence of linkage. Arthritis Rheum. 2004, 50:2920-2930.

1. A method of determining if a tissue is undergoing altered growth comprising measuring c19orf10 expression levels in the tissue wherein c19orf10 expression levels below a threshold level indicates that the tissue is undergoing altered growth.

2. A method of monitoring changes in a tissue comprising determining c19orf10 expression level in the tissue at a first time point; and determining c19orf10 expression level in the tissue at a second time point.

3. A method of treating a joint comprising administering to the joint an effective amount of a c19orf10 peptide.
